(19) United States
(12) Patent Application Publication
(10) Pub. No.: US 2024/0350151 A1
(43) Pub. Date: Oct. 24, 2024

(54) Sternal Bridge

(71) Applicant: Amrita Vishwa Vidyapeetham, Tamil Nadu (IN)

(72) Inventors: Mahesh Kappanayil, Kerala (IN); Harikrishnan Anil Maya, Kerala (IN); Balaji Srimurugan, Tamil Nadu (IN); Sarin Xavier, Kerala (IN)

(73) Assignee: Amrita Vishwa Vidyapeetham, Tamil Nadu (IN)

(21) Appl. No.: 18/674,500

(22) Filed: Jun. 26, 2024

(30) Foreign Application Priority Data
Nov. 30, 2023 (IN) 202341081246

(51) Int. Cl. A61B 17/17; A61B 17/02

(52) U.S. Cl. CPC A61B 17/1789; A61B 2017/0243

(57) ABSTRACT

A sternal bridge has a contiguous panel having a first length and a first height, a first U-shaped notch of a second height less than the first height and a first depth on one end of the contiguous panel, a second U-shaped notch of the second height and the first depth on an opposite end of the contiguous panel, a concave shape on a lower surface of the contiguous panel, and a first through hole passing from the lower surface of the panel through both sides of the first U-shaped notch, and a second through hole passing from the lower surface of the panel through both sides of the second U-shaped notch, the first and second through holes enabling the sternal bridge to be sutured onto the chest wall on either side.

### CROSS REFERENCE TO RELATED APPLICATIONS

The present application claims priority to India patent application No. 202341081246 filed 30 Nov. 2023. All disclosure of the parent case is incorporated herein at least by reference.

### BACKGROUND OF THE INVENTION

### 1. Field of the Invention

The present invention is in the technical field of surgical devices and pertains more particularly to a device for keeping a sternal opening secure in a cardiac procedure.

### 2. Description of Related Art

Heart surgery is usually performed through an incision in the midline of the chest (median sternotomy) which gives fair access to the heart and to other organs within the thoracic cavity.

Open heart surgeries are complex surgeries, wherein a sternum (breast-bone) is cut longitudinally and retracted during the surgery to provide access to the heart. Normally the sternum is sutured back with steel wires immediately after the surgery.

However, in some difficult, long and complicated surgeries, there is excessive tissue swelling (oedema) of the heart and surrounding tissues making it impossible for the sternum to be closed immediately as to do so would put pressure on the heart and circulation. In such cases, the sternum needs to be left open temporarily, such as for a few hours or days depending upon the condition of the patient. This is particularly true in the case of complex pediatric heart surgeries, especially in newborns and young infants.

Conventionally, in such cases, the sternum is wedged open by inserting a barrier such as a barrel of a syringe or other object repurposed, between the cut edges of the sternum to keep it open. This procedure is far from ideal.

By a sternal bridge in this specification is meant an apparatus purposed to keep the sternal opening open for a needed time after surgery. By the term “retractor” is meant a mechanical device, known in the art, for urging the sternal opening open after the sternum is surgically divided.

There are various patents and non-patent literature which disclose retractors which are used in cardiac surgery. Some examples are given below:

WO-2001080725-A1 discloses a device for annular fitting of a mechanical prosthesis of the heart valve, having a body made in the form of a ring, characterized in that it includes clamps equipped with spouts and hooks with sawtooth protrusions for engagement with the spouts of the clamps, while rectangular holes are made in the body for clamps and hooks. The hooks are installed with a possibility of alternately retracting to expand the seat of the mechanical prosthesis of the heart valve by moving the clamps with spouts towards the sawtooth protrusions of the hooks and loosening after completion of the annular landing.

Another patent, U.S. Pat. No. 690,501A1, teaches a retractor with devices that allow a surgeon to control the amount of retractor force. The retractor may include a first blade and a second blade. The second blade can be moved by a gear mechanism. A torque measuring device, force measuring device and/or slip clutch may be coupled to the gear mechanism. The torque measuring device may have a readout that displays the amount of torque being applied by the surgeon to the retractor. The readout provides accurate feedback that allows the surgeon to gauge the amount of force being applied to the patient. Likewise, the force measuring device may provide a visual indication of the actual force being applied by the retractor onto the patient. The slip clutch may actuate at a threshold torque to prevent an excessive exertion of force on a patient.

The various retractors disclosed in the existing state of art are bulky and have multiple components which make a process of using the device cumbersome. The bulky nature of the devices in the existing state of art adds extra expenditure to manufacturing and processing.

What is clearly needed is a sternal bridge which has a simple construction and is easy to handle, clean and sterilize.

### BRIEF SUMMARY OF THE INVENTION

In one embodiment of the invention a sternal bridge is provided, comprising a contiguous panel having a first length and a first height, a first U-shaped notch of a second height less than the first height and a first depth on one end of the contiguous panel, a second U-shaped notch of the second height and the first depth on an opposite end of the contiguous panel, a concave shape on a lower surface of the contiguous panel, and first through hole passing from the lower surface of the panel through both sides of the first U-shaped notch, and a second through hole passing from the lower surface of the panel through both sides of the second U-shaped notch, the first and second through holes enabling the sternal bridge to be sutured onto the chest wall on either side.

In one embodiment the sternal bridge is made from a bio-safe material. Also, in one embodiment the bio-safe material is one of stainless-steel, titanium or plastic. Also, in one embodiment the sternal bridge is manufactured by 3D printing. And in one embodiment the sternal bridge is manufactured by injection molding or machining.

In an alternative embodiment an adjustable sternal bridge is provided, comprising a first contiguous panel having a first length from a first end and a first height, a first U-shaped notch of a second height less than the first height and a first depth from the first end of the first contiguous panel, the first depth less than the first length, a second contiguous panel having the first length from a first end of the second contiguous panel and the first height, a second U-shaped notch of the second height and the first depth from a second end of the second contiguous panel, the second U-shaped notch facing opposite the first U-shaped notch, an extension having a rectangular cross-section extending from the first contiguous panel toward the second contiguous panel and into a rectangular passage in the second contiguous panel, a plurality of holes of a first diameter extending upward through the extension, sequentially spaced along a length of the extension, a retainer hole of a diameter greater than the first diameter downward into the second contiguous panel near the first end of the second contiguous panel, a retainer having a vertical post of a diameter less than the retainer hole, extending downward from a handle joined to the post, such that the post may be passed into the retainer hole and through one of the plurality of holes in the extension, fixing the first and the second contiguous panels at a fixed distance apart, a first through hole passing upward from a lower surface through both sides of the first U-shaped notch, and a second through hole passing from the lower surface through both sides of the second U-shaped notch, the first and second through holes enabling the adjustable sternal bridge to be sutured onto a portion of a chest wall on either side. The post may be withdrawn by the handle from the retainer hole and the first and second contiguous panels may be repositioned at a different distance apart, and the post returned through a different hole in the extension, resetting the distance between the first and second contiguous panels.

In one embodiment the adjustable sternal bridge is made from a bio-safe material. Also, in one embodiment the bio-safe material is one of stainless-steel, titanium or plastic. In one embodiment the adjustable sternal bridge is manufactured by 3D printing. And in one embodiment the adjustable sternal bridge is manufactured by injection molding or machining.

### BRIEF DESCRIPTION OF THE SEVERAL VIEWS OF THE DRAWINGS

FIG. 1A is a front elevation view of a simple, contiguous sternal bridge in an embodiment of the invention.

FIG. 1B is a side elevation view of the sternal bridge of FIG. 1A.

FIG. 1C is an end elevation view of the sternal bridge of FIG. 1A in an embodiment of the invention.

FIG. 2A is a front elevation view of an adjustable sternal bridge in an embodiment of the invention.

FIG. 2B is a side elevation view of the adjustable sternal bridge of FIG. 2A in an embodiment of the invention.

FIG. 2C is an end elevation view of the adjustable sternal bridge of FIG. 2A in an embodiment of the invention.

### DETAILED DESCRIPTION OF THE INVENTION

The present invention discloses devices in different embodiments intended to be used for keeping the sternum open after complicated heart surgery in a secure, safe and effective manner.

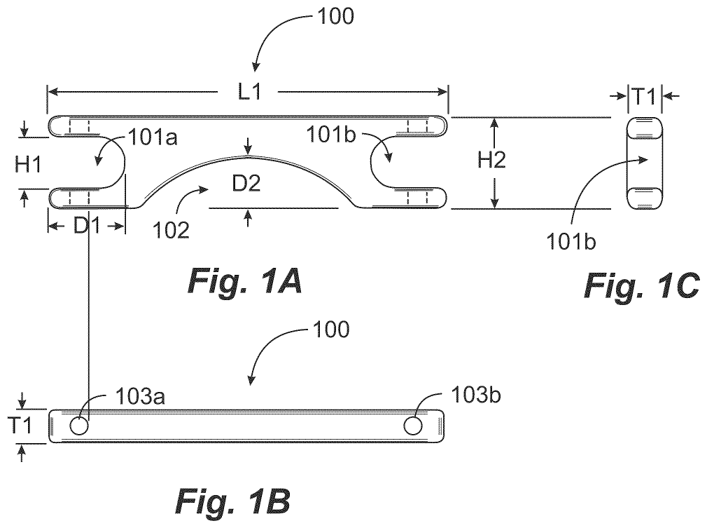

FIG. 1A is a front elevation view of a simple, contiguous sternal bridge 100 in an embodiment of the invention. Device 100 has a flat upper surface and a gently concave shape 102 on a lower surface which extends to elongated dual U-shaped notches 101a and 101b on either side. Concave shape 102 of the lower surface provides room, with the device installed, for underlying swollen tissues and the heart. Notches 101a and 101b on installation engage the cut edges of the sternum firmly and steadily.

FIG. 1B is a side elevation view of the sternal bridge of FIG. 1A, and illustrates two holes 103a and 103b which pass through the material on each side of the U-shaped notches, and may be used to suture the sternal bridge to the chest wall on either side. FIG. 1C is an end elevation view of the sternal bridge of FIG. 1A in an embodiment of the invention, illustrating the U-shaped notch 101b.

The device may be manufactured in different sizes for children of different sizes.

The sternal bridge device in different embodiments has a form-factor that fulfils an intended purpose of keeping the divided sternum wedged open. Notches 101a and 101b at either end engage the cut-edges of the divided sternum firmly yet non-traumatically.

Sternal bridges according to embodiments of the invention may be manufactured in different sizes. Size depends on the distance that the surgeons want to cleave the resected sternum. Based on this distance, the varying sizes of sternal bridge can part the resected sternum in a range from 15 mm to 40 mm.

In one embodiment the sternal bridge is approximately 55 mm long and has a radius of curvature of 20 mm. The curvature of the sternal bridge may be regular, that is, with a single radius of curvature or it may have multiple radii of curvature along its length to provide variation in the angle of the notches with respect to one another. The dual notches 101a and 101b of the sternal bridge in one embodiment are 10.5 mm long.

The sternal bridge in different embodiments may be manufactured for either single use, or for multiple uses after sterilization.

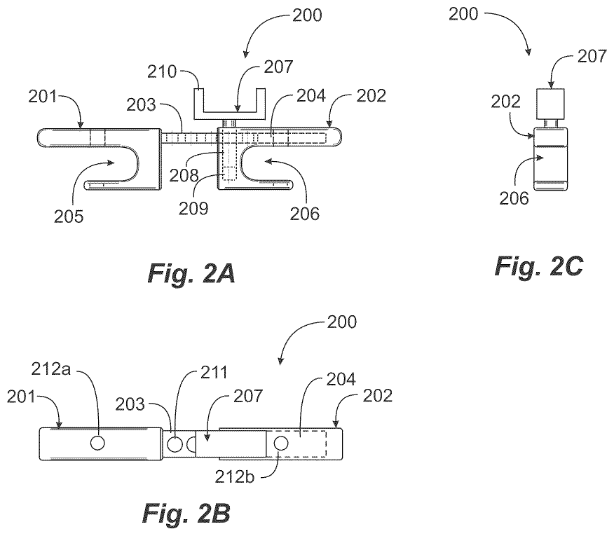

FIGS. 2A, 2B and 2C illustrate an alternative sternal bridge that is adjustable for the breadth of opening of the divided sternum. FIG. 2A is a front elevation view of an adjustable sternal bridge 200 comprising two principle parts, 201 and 202. Part 201 has a U-shaped notch 205 for engaging one side of the divided sternum. A rectangular extension 203 from part 201 engages a rectangular cavity 204 in part 202, such that parts 201 and 202 may be placed apart at different dimensions.

Extension 203 has a sequential series of holes 211 along its length, evenly spaced, which may be aligned with a vertical hole 209 in part 202, such that a retainer 207 having a post 208 and a handle 210 may be engaged with the post 208 into hole 209 through extension 203 to retain the parts 201 and 202 at different dimensions of separation.

This adjusting feature provides flexibility to decide the distance of separation required between the cut-edges of the divided sternum depending upon specific circumstances. This also enables a choice of re-adjusting and fine-tuning the separation distance (either reduction or increase) during the course of the care, using the same device instead of having to change to next fixed-size devices.

Dual notches 205 and 206 engage the cut-edges of the divided sternum firmly yet non-traumatically, with precisely placed holes 212a and 212b through the sides of the notches to allow the sternal bridge to be sutured onto the chest wall on either side.

The sternal bridge in different embodiments may be manufactured in a wide range of bio-safe materials including stainless-steel, titanium and a range of suitable plastics or similar materials. The device may be manufactured by various means such as, but not limited to 3D printing, injection molding or machining. Both resin and plastic, depending on the 3D printing method, may be used for manufacturing the device.

The skilled artisan will be aware that the embodiments illustrated and described in this application are entirely exemplary and are not limiting to the scope of the invention, which is limited only by the claims that follow.

1. A sternal bridge, comprising:
a contiguous panel having a first length and a first height;
a first U-shaped notch of a second height less than the first height and a first depth on one end of the contiguous panel;
a second U-shaped notch of the second height and the first depth on an opposite end of the contiguous panel;
a concave shape on a lower surface of the contiguous panel; and
a first through hole passing from the lower surface of the panel through both sides of the first U-shaped notch, and a second through hole passing from the lower surface of the panel through both sides of the second U-shaped notch, the first and second through holes enabling the sternal bridge to be sutured onto the chest wall on either side.

2. The sternal bridge of claim 1 made from a bio-safe material.

3. The sternal bridge of claim 2 wherein the bio-safe material is one of stainless-steel, titanium or plastic.

4. The sternal bridge of claim 1 manufactured by 3D printing.

5. The sternal bridge of claim 1 manufactured by injection molding or machining.

6. An adjustable sternal bridge, comprising:
a first contiguous panel having a first length from a first end and a first height;
a first U-shaped notch of a second height less than the first height and a first depth from the first end of the first contiguous panel, the first depth less than the first length;
a second contiguous panel having the first length from a first end of the second contiguous panel and the first height;
a second U-shaped notch of the second height and the first depth from a second end of the second contiguous panel, the second U-shaped notch facing opposite the first U-shaped notch;
an extension having a rectangular cross-section extending from the first contiguous panel toward the second contiguous panel and into a rectangular passage in the second contiguous panel;
a plurality of holes of a first diameter extending upward through the extension, sequentially spaced along a length of the extension;
a retainer hole of a diameter greater than the first diameter downward into the second contiguous panel near the first end of the second contiguous panel;
a retainer having a vertical post of a diameter less than the retainer hole, extending downward from a handle joined to the post, such that the post may be passed into the retainer hole and through one of the plurality of holes in the extension, fixing the first and the second contiguous panels at a fixed distance apart;
a first through hole passing upward from a lower surface through both sides of the first U-shaped notch, and a second through hole passing from the lower surface through both sides of the second U-shaped notch, the first and second through holes enabling the adjustable sternal bridge to be sutured onto a portion of a chest wall on either side;
wherein the post may be withdrawn by the handle from the retainer hole and the first and second contiguous panels may be repositioned at a different distance apart, and the post returned through a different hole in the extension, resetting the distance between the first and second contiguous panels.

7. The adjustable sternal bridge of claim 6 made from a bio-safe material.

8. The adjustable sternal bridge of claim 7 wherein the bio-safe material is one of stainless-steel, titanium or plastic.

9. The adjustable sternal bridge of claim 6 manufactured by 3D printing.

10. The adjustable sternal bridge of claim 6 manufactured by injection molding or machining.
